(19) United States
(12) Patent Application Publication
(10) Pub. No.: US 2026/0266935 A1
(43) Pub. Date: Sep. 10, 2026

(54) METHOD FOR ASCERTAINING CONSISTENCY INFORMATION FOR A LOCAL COIL

(71) Applicant: Siemens Healthineers AG, Forchheim (DE)

(72) Inventor: Thomas BECK, Dormitz (DE)

(73) Assignee: Siemens Healthineers AG, Forchheim (DE)

(21) Appl. No.: 19/554,728

(22) Filed: Mar. 3, 2026

(30) Foreign Application Priority Data
Mar. 4, 2025 (DE) 10 2025 108 080.1

(51) Int. Cl. G01R 33/341; A61B 5/055; G01R 33/54

(52) U.S. Cl. CPC G01R 33/341; A61B 5/055; G01R 33/543; G01R 33/546

(57) ABSTRACT

One or more example embodiments relates to a method for ascertaining consistency information for a local coil of a magnetic resonance apparatus. The magnetic resonance apparatus is configured to ascertain medical image data of a predetermined region of the body of a patient through an imaging examination via the local coil. The method comprises acquiring measurement information of the local coil. The measurement information comprises an actual position of the local coil. The method comprises ascertaining positioning information. The positioning information comprises a required position of the local coil. The method comprises comparing the measurement information with the positioning information. The method comprises ascertaining the consistency information with the aid of a result of the comparison.

### CROSS-REFERENCE TO RELATED APPLICATION(S)

The present application claims priority under 35 U.S.C. § 119 to German Patent Application No. 10 2025 108 080.1, filed Mar. 4, 2025, the entire contents of which are incorporated herein by reference.

### FIELD

One or more example embodiments relates to a local coil of a magnetic resonance unit. For example, one or more example embodiments relates to the positioning of a local coil on a part of a patient's body for a magnetic resonance examination via a magnetic resonance unit.

### RELATED ART

Medical imaging, in particular magnetic resonance tomography (MRT), is a major diagnostic means for examination of parts of patients' bodies, such as extremities of the human body. A problem that frequently occurs during imaging of parts of the body and in particular of extremities lies in the confusion between sides of the body. In particular this can occur with those parts of the body that are typically present more than once and are present both on the left side and also on the right side of the human body. This can lead to the incorrect region of the body or part of the body being displayed. The consequence of these confusions can for example be the necessity for carrying out a repeat imaging procedure. Repeat imaging procedures in turn can lead to lost time and money.

As well as the incorrect positioning of the local coil on the patient and/or within the magnetic resonance apparatus, inconsistencies in the pre-registration of the control software of the magnetic resonance apparatus (also referred to as the MRT operating interface and/or patient registration) and/or of a radiology information system (also referred to as examination planning software) can occur.

The use of dedicated local coils or coils able to be used flexibly for imaging of specific parts of the body is known. With parts of the body that occur both on the left side and the right side of the body, the person conducting the examination must position the local coil on the correct side by hand before the imaging examination and select the correct side of the body in the MRT operating interface, or must check the pre-registration. Despite these checking measures undertaken by the person conducting the examination, errors continue to occur, in particular confusion between sides, which adversely affect the diagnostic workflow or slow it down.

### SUMMARY

Example embodiments improve the efficiency of imaging examinations, and such as the preparation for the imaging examination via a magnetic resonance apparatus. For example, one or more example embodiments ensures a correct positioning of the local coil for an imaging examination via a magnetic resonance apparatus. Moreover, it can be seen as an object of the invention to reduce positioning errors of the local coils of a magnetic resonance apparatus and/or bring them to the attention of a person conducting the examination.

### BRIEF DESCRIPTION OF THE DRAWINGS

Further advantages, features and details emerge from the exemplary embodiments described below, as well as with the aid of the drawings. Parts that correspond to one another are labeled with the same reference characters in all figures. Modifications mentioned in this context can each be combined with one another to form new forms of embodiment.

In the figures:

FIG. 1 shows a flow diagram of a method for ascertaining consistency information for a local coil of a magnetic resonance apparatus in accordance with a preferred form of embodiment with an optional method step for provision of the consistency information,

FIG. 2 shows a flow diagram of a method for ascertaining consistency information for a local coil of a magnetic resonance apparatus comprising a number of optional method steps in accordance with a preferred form of embodiment,

FIG. 3 shows a data flow diagram of a method for ascertaining consistency information for a local coil of a magnetic resonance apparatus in accordance with one form of embodiment,

FIG. 4 shows a schematic diagram of a form of embodiment of a magnetic resonance apparatus in accordance with one form of embodiment, and

FIG. 5 shows a schematic diagram of a form of embodiment of a local coil in accordance with one form of embodiment.

### DETAILED DESCRIPTION

In accordance with one or more example embodiments a method for ascertaining consistency information for a local coil of a magnetic resonance apparatus is proposed. The method comprises an acquisition of measurement information of the local coil. The method comprises acquiring positioning information. The method comprises ascertaining the consistency information by a comparison of the measurement information with the positioning information. The measurement information comprises an actual position of the local coil. The positioning information comprises a required position of the local coil. The magnetic resonance apparatus is embodied to ascertain medical image data of a predetermined region of a patient's body through an imaging examination via the local coil.

The magnetic resonance apparatus is usually embodied for an acquisition of medical and/or diagnostic image data of a patient. In particular the magnetic resonance apparatus can be embodied for recording of medical image data of a predetermined region of the body of a patient via a local coil and/or a number of local coils. The magnetic resonance apparatus can comprise one or more local coils. The magnetic resonance apparatus, in particular a control and/or processing unit of the magnetic resonance apparatus, can be connected to one and/or to a number of local coils for signal transmission.

The magnetic resonance apparatus further typically comprises a magnet unit, a patient table and a control unit. The magnet unit encloses a patient receiving area delimited by an inner wall of the magnet unit and can comprise a whole body coil. The patient table is embodied movably in the patient receiving area. In particular the part of the patient's body is supported in the magnet opening, from which imaging and/or spectroscopic magnetic resonance signals are to be acquired with the magnetic resonance apparatus. In particular at least a part of the patient is supported in an isocenter of the magnetic resonance apparatus, in particular of a main magnetic field. By way of the irradiation of the radio frequency transmit pulses radio frequency fields are usually created during the magnetic resonance examination, with which nuclear spins in the patient, in particular in a region of the body, are diverted from a rest position. By way of a subsequent relaxation, imaging and/or spectroscopic magnetic resonance signals are generated, which are received by one or more receive antennas of the magnetic resonance apparatus and can be used for reconstruction of magnetic resonance images or for spectroscopy. In particular the whole body coil, which is permanently integrated into a magnet unit of the magnetic resonance apparatus, comprises at least one receive antenna and/or a transmit antenna.

While the whole body coil is embodied in particular for recording of large surface areas of a patient's body, a local coil can be used as an additional receiver and/or detector in order to acquire image data from specific parts of the body. The local coil is embodied to be placed directly on and/or in the immediate vicinity of the part of the body to be examined, in order to receive signals of the tissue of the part of the body through one or more radio frequency receive antennas of the local coil. Various types of local coil are known for different parts of the body, for example orthopedic coils for the examination of joints such as the knee, the shoulder, the wrist or the ankle. Local coils are typically adapted in their shape and dimensions to the respective anatomy of the part of the body to be examined. What is more, coils for flexible use on various parts of the body are known.

In particular an imaging examination can be carried out via the magnetic resonance apparatus using an imaging protocol. During the imaging examination the patient is typically located entirely or in part in the patient receiving area. In particular the patient can be positioned during the imaging examination on a patient support surface of the patient table. For creation of imaging and/or spectroscopic magnetic resonance signals during an imaging examination, radio frequency transmit pulses in accordance with a measurement protocol, in particular a magnetic resonance sequence, are irradiated with one or more transmit antennas, into the patient receiving area, in particular into the patient, in particular into a part of the body. The imaging examination can likewise also be referred to as a magnetic resonance examination.

The medical image data can in particular comprise data and/or information that will be created during a medical imaging examination via the magnetic resonance apparatus, in particular with the aid of an imaging protocol. The medical image data can for example contain information about the intensity and/or phase of magnetic resonance signals that are emitted from a patient, for example. The medical image data can in particular comprise data in various data formats, for example raw data, K-space data or image space data. The medical image data can in particular be used as input data for reconstruction of medical images. The medical image data can in particular comprise information, in particular reconstructed image data, about the anatomical and/or functional characteristics of the patient being examined. The medical image data can in particular comprise signals acquired during a medical imaging examination.

A region of the body refers in particular to a part area and/or a part region of a body of a patient. A region of the body can in particular be a region of the body and/or part of the body of the patient. In the context of example embodiments the term “region of the body” refers in particular to joints and/or extremities, such as for example knee, ankle, shoulder, elbow or wrist. The predetermined region of the body is preferably the region of the body from which image data is to be ascertained via the imaging examination. Preferably the patient has a further, in particular symmetrical, region of the body corresponding to the predetermined region of the body.

The measurement information can in particular comprise information identifying the actual position of the local coil. In particular the measurement information can be ascertained via a device suitable for determining the position of the local coil. In particular the measurement information can be ascertained via a measuring instrument, a sensor unit, a position measurer. The actual position can in particular identify the position of the local coil at a particular point in time. The measurement information of the local coil can preferably be acquired at two points in time at two locations: in a starting position of the patient table (on which the patient is positioned for the imaging examination and which can preferably be moved into the magnetic resonance apparatus) before the imaging examination and/or within the magnetic resonance apparatus (in particular with the patient in the examination position for the imaging examination) in particular immediately before the imaging examination. In particular the acquisition of the measurement information of the local coil can take place while a patient table is being moved into the magnetic resonance apparatus. In particular the measurement information can identify a positioning (actual position) of the local coil in three spatial directions. Preferably the measurement information can describe the (ideally actual) spatial location of the actual position of the local coil. The measurement information can in particular comprise information about the region of the body around which and/or on which the local coil is placed. In other words, it can be determined via the acquisition of the measurement information on which region of the body the local coil is placed.

Preferably the measurement information can comprise a three-dimensional (for example in x, y and z) and/or two-dimensional description (for example in x and y) of a measurement point starting from a reference object, in particular a reference point, via a coordinate system. In particular the measurement information can comprise the actual position of the local coil in relation to a coordinate system and/or reference point. The reference point (for example zero point of the coordinate system) can preferably be an isocenter of the magnetic resonance apparatus and/or a reference point of the patient table (for example an end of the table and/or table midpoint). The reference point can preferably be an unchanging point of the magnetic resonance apparatus. The measurement information can in particular be a distance and/or path. In particular the measurement information can be a height specification and/or a length value of a path (for example in x, y and/or z direction) of a coordinate system. In particular the measurement information can be ascertained by a sensor unit and be transferred/output to a system and/or user interface.

The positioning information can in particular comprise information identifying the required position of the local coil. In particular the positioning information can be dependent on the region of the body of the patient to be examined and/or predetermined. In other words the positioning information can in particular comprise information about the region of the body around which and/or on which the local coil is to be placed. In other words the positioning information can identify the required position of the local coil on the predetermined region of the body. In particular the positioning information can be ascertained from information of an imaging examination preparation. Information of the imaging examination preparation can comprise a patient registration and/or information from a database and/or patient file. For example it can be determined by an operator that a left wrist is to be examined by way of an imaging examination as a predetermined region of the body of the patient lying on their back and feet first. From this information (of the predetermined region of the body) the required position of the local coil can be determined. The required position can in particular identify a predetermined position of the local coil. The required position can for example identify which region of the body from a plurality of regions of the body and/or parts of the body is to be recorded. In other words the required position of the local coil can identify whether a left or right hand of the patient is to be recorded. In other words the side of the body to be examined, in particular the predetermined region of the body, can be determined by the positioning information, in particular the required position of the local coil.

In particular the positioning information can identify a positioning (required position) of the local coil in three spatial directions. Preferably the positioning information can describe the ideal spatial position of the local coil in the magnetic resonance apparatus. Preferably the positioning information can comprise a three-dimensional (for example in x, y and z) and/or two-dimensional description (for example in x and y) of a positioning point starting from a reference object, in particular a reference point, via a coordinate system. In particular the positioning information can comprise the required position of the local coil in relation to a coordinate system and/or to a reference point. The positioning information can in particular be a distance and/or path. In particular the positioning information can be a height specification and/or a length value of a path (for example in x, y and/or z direction) of a coordinate system. In particular the positioning information can be ascertained and/or transferred/output by a processing unit and/or system and/or user interface.

The comparison of the measurement information with the positioning information can in particular comprise the ascertaining of a difference and/or a correlation. In particular a comparison of the measurement information with the positioning information by way of a determination of deviations that are possible and/or have occurred, errors and/or peculiarities of the actual position of the local coil with regard to the required position of the local coil can be identified.

The consistency information can in particular comprise difference information and/or correlation information of the measurement information with the positioning information. The comparison information can in particular comprise information containing the result of a comparison of the required position of the local coil with the actual position of the local coil. In particular the consistency information can be information about deviations, errors and peculiarities that are possible and/or have occurred in the actual position of the local coil mapped by the measurement information compared to a required position of the local coil mapped by the positioning information. In particular the consistency information can comprise information about a possible deviation and/or one that has occurred, errors of a predetermined region of the body depicted by the positioning information and an actual region of the body at which the local coil is arranged depicted by the measurement information. In other words the consistency information can preferably identify whether the local coil is positioned on the predetermined region of the body (and not on a corresponding, in particular symmetrical, region of the body). In particular the consistency information can comprise information that can make it clear to an operator of the magnetic resonance apparatus whether the right region of the body, in particular in relation to the side (of the body), is being depicted by measurement information and/or positioning information.

The consistency information can in particular comprise one or more numerical values and/or character strings, for example words. The consistency information can in particular comprise a “true/false” distinction. For example the consistency information can comprise a degree of deviation of the actual position from the required position of the local coil. For example the consistency information can comprise information for confirming a region of the body and/or the positioning of the local coil.

Advantageously, by way of the method for ascertaining consistency information, the efficiency and accuracy of imaging examinations via a magnetic resonance apparatus can be. The correct positioning of the local coil can advantageously be ensured by way of the proposed method. This can minimize mispositionings of the local coil and imaging errors associated therewith. Moreover, the likelihood of positioning errors of parts of the body is reduced and the person conducting the examination is informed about possible inconsistencies. Thus the quality of the medical image data can be enhanced. The method can involve considerable time and cost savings for patient and medical personnel and/or facilities, since repeat examinations can be avoided. Overall the method can make possible a more precise diagnosis and better decisions by medical personnel in medical imaging processes.

In accordance with one or more example embodiments of the invention the ascertaining of the positioning information comprises a determination of the required position of the local coil with the aid of the predetermined region of the body of the patient.

The positioning information can comprise information defined by an operator about a region of the body of a patient to be examined via the medical imaging apparatus. The predetermined region of the body of the patient preferably corresponds to the region of the body to be examined from an examination planning. The examination planning can be provided by an operator, a memory unit and/or a database to the magnetic resonance apparatus. Preferably the magnetic resonance apparatus, in particular a processing unit of the magnetic resonance apparatus, can be embodied to ascertain a required position of the local coil automatically from a predetermined region of the body of the patient and optionally display it to an operator. For example an operator can determine the left knee of a patient as the region of the body to be examined. The magnetic resonance apparatus can for example determine the positioning of the local coil around the left knee of the patient and optionally display it to an operator via an output unit.

Advantageously, through the ascertaining of the required position of the local coil with the aid of the predetermined region of the body, possible deviations of the positioning of the local coil are minimized and in particular the consistency and/or efficiency of the imaging is thus improved. This leads to a correct positioning of the local coil and to consistent, high-quality medical image data.

In accordance with one or more example embodiments of the invention the method moreover comprises an ascertaining of medical image data of the predetermined region of the body of the patient by an imaging examination. The imaging examination is undertaken as a function of the consistency information.

In other words the imaging examination is advantageously undertaken via the magnetic resonance apparatus only for a positive result of the comparison of the measurement information with the positioning information. In other words the imaging examination is undertaken via the magnetic resonance apparatus only when the consistency information matches predetermined information. For example the consistency information can have a binary value (“0 or 1”) and an imaging examination can only be undertaken with a predetermined value, for example “1”.

Advantageously it can be ensured by this that an imaging examination can only take place with a correct positioning of the local coil at the predetermined region of the body.

In accordance with one or more example embodiments of the invention the method moreover comprises an ascertaining of a patient registration. The patient registration comprises parameters for an imaging examination. The ascertaining of the positioning information comprises a determination of the required position of the local coil with the aid of the patient registration.

The patient registration can for example comprise a Radiology Information System (RIS) and/or preferably examination planning software of the magnetic resonance apparatus. In particular the patient registration can comprise data about the patient, such as age, body size, diagnosis. In particular the patient registration can comprise the parameters that are needed for the imaging examination and/or which must be predetermined. For example the patient registration can comprise information about the type of the local coil to be used. The patient registration can be determined by information entered by the operator. The patient registration can in particular be determined automatically, at least in part (one or more parameters), from historical patient data. The ascertaining of the patient registration can be undertaken by a processing unit of the magnetic resonance apparatus. The patient registration can be provided to an operator by an output unit of the magnetic resonance apparatus.

A parameter for the imaging examination can in particular comprise information for control of a medical imaging apparatus and/or imaging parameters, in particular a measurement protocol for carrying out a medical imaging examination. The parameter can particularly comprise information about an execution of an imaging examination via a magnetic resonance apparatus. In particular a number of parameters can determine a measurement protocol and/or a measurement sequence. For example the parameters (also imaging parameters) can comprise a slice spacing, number of slices, slice thickness, a turbo factor and/or a flip angle. In other words the parameters can in particular comprise information about a type of the examination and/or imaging apparatus, parameters, values, setting information and/or a protocol of the medical imaging examination.

Advantageously the ascertaining of the patient registration and in particular of the parameters for the imaging examination associated therewith enables a precise determination of the required position of the local coil to be made possible. This can lead to an improved positioning of the local coil and thus to a more efficient execution of the imaging examination. Advantageously the patient registration can clarify to the operator the required position of the local coil and/or parameters of the planned imaging examination. Advantageously the positioning of the local coil can be made easier for the operator by this. Advantageously the efficiency of an imaging examination preparation can moreover be improved.

In accordance with one or more example embodiments of the invention the patient registration is ascertained with the aid of information from a hospital and/or radiology information system.

A hospital and/or radiology information system (also HIS and/or RIS) can in particular comprise an information system for the administration and/or organization of information in hospitals and/or other healthcare facilities. A hospital and/or radiology information system can in particular make possible the acquisition, storage and/or administration of patient data, medical image data, schedule planning, billing, electronic health records (EHR), laboratory information. In particular the hospital and/or radiology information system can be embodied as a database. In particular the hospital and/or radiology information system can comprise a database. In particular the hospital and/or radiology information system can be connected via a network to other systems and/or apparatus of a healthcare facility, in particular to a magnetic resonance apparatus.

The ascertaining of the patient registration with the aid of information from a hospital and/or radiology information system can in particular be undertaken by a processing unit of the magnetic resonance apparatus. In particular the processing unit can be connected to a database (of the hospital and/or radiology information system). In particular the processing unit can be embodied to search the hospital and/or radiology information system for predefined information for the ascertaining of the patient registration. In particular the ascertaining of the patient registration can be undertaken via a trained function. In particular the trained function can be embodied to reconcile a predetermined region of the body with information from the hospital and/or radiology information system and/or to determine a required position of the local coil.

Advantageously an automatic ascertaining of the patient registration from a hospital and/or radiology information system can make possible a precise and automatic patient data acquisition. Through this the data stored in a hospital and/or radiology information system can advantageously be taken into account in the ascertaining of the consistency information and thereby errors in the creation of medical image data can be reduced.

In accordance with one or more example embodiments of the invention the method moreover comprises a provision of the consistency information to an operator of the magnetic resonance apparatus.

The provision of the consistency information to an operator of the magnetic resonance apparatus comprises in particular a display, transmission, storage or processing of the consistency information. The consistency information can for example be output and/or clarified to an operator via a display unit of the magnetic resonance apparatus. The consistency information can for example be stored in a database, a processing unit and/or control unit, be transmitted to said unit and/or be further processed via said unit.

Advantageously the result of the comparison of the measurement information with the positioning information can be clarified to the operator by the provision of the consistency information. Through this it can be made possible in particular for an operator to recognize a discrepancy between the actual positioning of the local coil and an intended positioning of the local coil.

In accordance with one or more example embodiments of the invention the method additionally optionally comprises an acquisition of a user input. With the aid of the user input there can be a change of the actual position of the local coil and/or a change of the positioning information, in particular of the patient registration.

The user input can be acquired for example via an output unit, in particular via a user interface comprised by the output unit. In particular the user input can be entered as a function of the consistency information provided. In particular the provision of the consistency information can comprise a generation of a presentation of the consistency information for display to an operator in a user interface. The presentation optionally comprises one or more reaction options. The generation of the presentation optionally comprises the creation of at least one reaction option from the consistency information. The provision of the presentation to the user can be undertaken in the user interface. The method can comprise a receipt, optionally via the user interface, of a user input, which aims to select a reaction option.

Advantageously the acquisition of a user input can make possible a simple correction of the actual position of the local coil and/or the changing of the positioning information, in particular of the patient registration.

In accordance with one or more example embodiments of the invention the provision of the consistency information comprises an output of a visual, haptic and/or acoustic notification signal to the operator of the magnetic resonance apparatus.

The output of a notification signal to the operator of the magnetic resonance apparatus can in particular come from a correspondingly embodied output unit, in particular of the magnetic resonance apparatus and/or the local coil. The output unit can in particular comprise a sound and/or light unit for creation of a sound and/or light signal. The output unit can be comprised by a unit, in particular of the user interface of the magnetic resonance apparatus. The output unit can be embodied to output an imaging parameter, the consistency information and/or a patient registration. For example the output unit can output a notification signal in the form of a flashing light signal. Preferably one output unit can be provided for a number of units, in particular a number of local coils, of the magnetic resonance apparatus.

The notification signal comprises a visual, haptic and/or acoustic signal recognizable for the operator, in particular a light signal and/or a sound signal. The notification signal can for example comprise a textual output and/or a graphical output via a user interface and/or output unit of the magnetic resonance apparatus. The notification signal can comprise a number of warning signals and/or notification signals. Depending on the consistency information, a notification signal can be output that can clarify a classification of incorrect positioning of the local coil for an operator of the magnetic resonance apparatus. For example a possible confusion between two parts of a patient's body to be examined (for example left elbow and right elbow) can be indicated by a loud warning sound as a notification signal. For example, depending on the positioning of the local coil, the frequency of a sound of the notification signal can be increased and/or reduced. Preferably the consistency information can be provided via a combination of a number of visual, haptic and/or acoustic notification signals.

The consistency information can advantageously be clarified by the notification signal to the operator in an efficient and user-friendly manner. It can be made visible to the operator in a reliable way through the notification signal that a specific item of consistency information is present. In particular, through the provision of the consistency information as a notification signal to the operator, the consistency information can advantageously additionally be clearly shown to a display for control of the magnetic resonance apparatus.

In accordance with one or more example embodiments of the invention the acquisition of the measurement information of the local coil is undertaken by a sensor unit comprised by the local coil.

In particular the sensor unit can comprise a Hall sensor. In particular the sensor unit can comprise a mechanical and/or electrical contact sensor. The measurement information can in particular comprise position information of at least one sensor unit.

A sensor unit (also sensor for short) is generally embodied to ascertain position information of an object. In particular the sensor unit can be embodied to ascertain position information as measurement information of at least one local coil. One and/or a number of sensors can form a sensor unit for ascertaining of the measurement information. The sensor unit can in particular ascertain, continuously and/or at specific intervals, the position information and/or output it. Furthermore the sensor unit can in particular only output one item of position information when the patient is positioned outside a magnet opening of the magnetic resonance apparatus and/or is moved into the magnet opening of the magnetic resonance apparatus, in particular via a patient table. The position information can comprise discrete information, for example specification of an angle and/or distance, about the spatial positioning of the local coil.

A sensor unit can in particular be embodied to detect a positioning of the local coil on the predetermined region of the body of the patient. For example the sensor unit can return a zero signal for positioning on another part of the patient's body. In particular the sensor unit can ascertain a position of the local coil on the patient table in the patient receiving area of the magnetic resonance apparatus. In particular the sensor unit can be connected to a processing unit of the magnetic resonance apparatus for data transmission. In particular the sensor unit can be embodied to provide measurement information to the processing unit.

A Hall sensor in general can determine the presence and the strength of a magnetic field through the so-called Hall effect. The Hall sensor can thus generate a voltage that is proportional to the magnetic field strength. This Hall voltage makes an ascertaining of the magnetic field intensity possible. From the magnetic field intensity ascertained there can be an ascertaining of position information, in particular of the measurement information. In other words, via the Hall sensor, there can be a precise position acquisition and movement acquisition of the local coil.

Electrical and/or mechanical contact sensors in general can detect a physical contact between components and convert it into an electrical signal. An electrical contact sensor typically closes a current circuit when two conductive surfaces come into contact. A mechanical contact sensor can typically detect mechanical movements, such as the closing of a switch. For example a local coil arranged on a patient table at a predetermined place can bring about a contact between the contact sensor and an assigned mating unit comprised by the patient table.

Advantageously the ascertaining of the measurement information of the local coil by a sensor unit comprised by the local coil makes possible an exact and robust determination of the position of the coil. A (continuous) checking and acquisition of the local coil position enables mispositionings of the local coil to be recognized and corrected at an early stage.

Moreover, in accordance with one or more example embodiments, a magnetic resonance apparatus for ascertaining medical image data of a predetermined region of the body of a patient via a local coil is proposed. The magnetic resonance apparatus is embodied to carry out a method as claimed in one of the previous aspects described.

In particular a magnet unit of the magnetic resonance apparatus can comprise a main magnet, a gradient coil unit, a radio frequency antenna unit. The main magnet of the magnet unit is preferably embodied for creation of a homogeneous (strong, constant) main magnetic field with a defined and/or specific magnetic field strength, such as for example with a defined and/or specific magnetic field strength of 3 T or 1.5 T or 0.55 T. The homogeneous main magnetic field is preferably arranged and/or to be found within a patient receiving area of the magnetic resonance apparatus. The magnet unit usually encloses the patient area (or patient receiving area), which is embodied for receiving a patient for a magnetic resonance examination. The gradient coil unit is preferably embodied for creation of gradient fields that are used for spatial encoding during imaging. The radio frequency antenna unit is preferably arranged permanently within the magnet unit and is designed and/or embodied for emitting an excitation pulse. To acquire the magnetic resonance signals the magnetic resonance apparatus preferably has local radio frequency coils (local coils), which are arranged around the region of the patient to be examined.

Moreover, the magnetic resonance apparatus can comprise a processing unit and/or output unit. The processing unit is in particular embodied for control and/or regulation of the medical magnetic resonance apparatus. The output unit is in particular embodied for provision of the consistency information to an operator, in particular in the form of a notification signal. The magnetic resonance apparatus can moreover comprise at least one sensor and/or sensor unit. The sensor can be embodied to detect an actual position of the local coil and/or to transfer measurement information comprising the actual position to the processing unit.

The advantages of the proposed magnetic resonance apparatus essentially correspond to the advantages of the proposed method. Features, advantages or alternative forms of embodiment/aspects of the method can likewise be transferred to the other claimed subject matter and vice versa.

In accordance with one or more example embodiments of the invention a sensor unit and/or sensor, in particular within the patient receiving area, can be arranged on the magnetic resonance apparatus, in particular on and/or in the inner wall of the magnet unit. The sensor unit can in particular be arranged outside the patient receiving area, in particular on an end face side and/or on a housing element of the magnet unit. The sensor unit can in particular be arranged at a reference point and/or relative to a reference point, for example the midpoint of the magnet opening or a predetermined point on the inner wall of the magnet unit. In other words the magnetic resonance apparatus can comprise a sensor unit for acquisition of measurement information of the local coil, which is not comprised by the local coil.

Advantageously a sensor unit comprised by the magnetic resonance apparatus makes possible a position determination of the local coil in particular independently of the local coil. In this way for example known local coils, in particular without a sensor unit, can be employed for position recognition.

Moreover, in accordance with one or more example embodiments, a local coil for ascertaining medical image data of a predetermined region of the body of a patient via a magnetic resonance apparatus is proposed. The local coil is embodied to carry out a method as claimed in one of the aspects previously described.

Local coils in general function according to the principle of electromagnetic induction. In particular local coils can receive signals that occur during a magnetic resonance examination, in particular magnetic resonance signals. During a magnetic resonance examination the main magnet of the magnetic resonance apparatus creates a strong, static magnetic field. Radio frequency (RF) pulses can be emitted, which in particular excite the hydrogen protons in the body of a patient, which can align themselves in parallel or antiparallel to the magnetic field. When the RF pulse is stopped, in particular the protons can return to their original state and, in doing so, emit RF signals. These signals can advantageously be received especially precisely by local coils.

To this end, the local coil typically comprises receive coils for receiving RF signals. Moreover, local coils can comprise preamplifiers in order in particular to improve the signal-to-noise ratio.

The local coil can moreover comprise at least one sensor and/or sensor unit. The sensor can be embodied to detect an actual position and/or measurement information of the local coil and to transfer measurement information comprising an actual position to the processing unit.

In accordance with one or more example embodiments of the invention the local coil can comprise an output unit. The local coil can be embodied to receive consistency information and, with the aid of the consistency information, to output a signal, in particular a notification signal, to an operator. For this purpose the local coil can comprise an output unit in the form of a light element and/or sound generation element. For example the local coil can comprise an LED light element that, depending on the consistency information ascertained, provides a predetermined color-coded notification signal to a user. In other words the consistency information in the form of a red flashing light element can be signaled as a notification signal at the local coil to an operator for a non-match between the measurement information and the positioning information.

Advantageously, the consistency information from/at the local coil can be made clear to an operator of the magnetic resonance apparatus by the output unit on the local coil. Advantageously in this way the operator can already be informed during positioning of the local coil about a possible incorrect position of the local coil.

In accordance with one or more example embodiments of the invention the local coil comprises a processing unit. The processing unit of the local coil is preferably embodied to carry out a method as claimed in one of the aspects previously described. The processing unit is in particular embodied for controlling and/or regulating the local coil. The processing unit of the local coil can be connected via a signal connection to a processing unit of the magnetic resonance apparatus.

Advantageously a processing unit comprised by the local coil can represent an additional autonomous unit (to the control unit of the magnetic resonance apparatus) for ascertaining the consistency information.

The advantages of the proposed local coil essentially correspond to the advantages of the proposed method and the proposed magnetic resonance apparatus. Features, advantages or alternative forms of embodiment/aspects of the method can likewise be transferred to the other claimed subject matter and vice versa.

A computer program product is further proposed that comprises a program and is able to be loaded directly into a memory of a programmable system control unit of a medical imaging apparatus and has program means, for example libraries and auxiliary functions, for carrying out a proposed method when the computer program product is executed in the system control unit of the magnetic resonance apparatus and/or local coil. The computer program product can in this case comprise software with a source code that still has to be compiled and linked or just has to be interpreted, or an executable software code that just still has to be loaded into the system control unit for execution.

The computer program product advantageously enables the proposed method to be carried out quickly, identically repeatedly and robustly. The computer program product is preferably configured so that the proposed method steps can be carried out via the system control unit. The system control unit in this case has the preconditions in each case such as for example a corresponding working memory, a corresponding graphics card or a corresponding logic unit, so that the respective method steps can be carried out efficiently.

The computer program product is stored for example on a computer-readable medium or is held on a network or server, from where it can be loaded into the processor of a local system control unit, which is directly connected to the magnetic resonance apparatus and/or local coil or can be embodied as a part of the magnetic resonance apparatus and/or of the local coil. Furthermore, control information of the computer program product can be stored on an electronically-readable data medium. The control information of the electronically readable data medium can be designed in such a way as to carry out a proposed method when the data medium is used in a system control unit of a magnetic resonance apparatus and/or local coil.

Examples of electronically readable data media are a DVD, a magnetic tape or a USB stick, on which electronically readable control information, in particular software, is stored. When this control information is read from the data medium and stored in a system control unit of the medical imaging apparatus, all proposed forms of embodiment of the method previously described can be carried out.

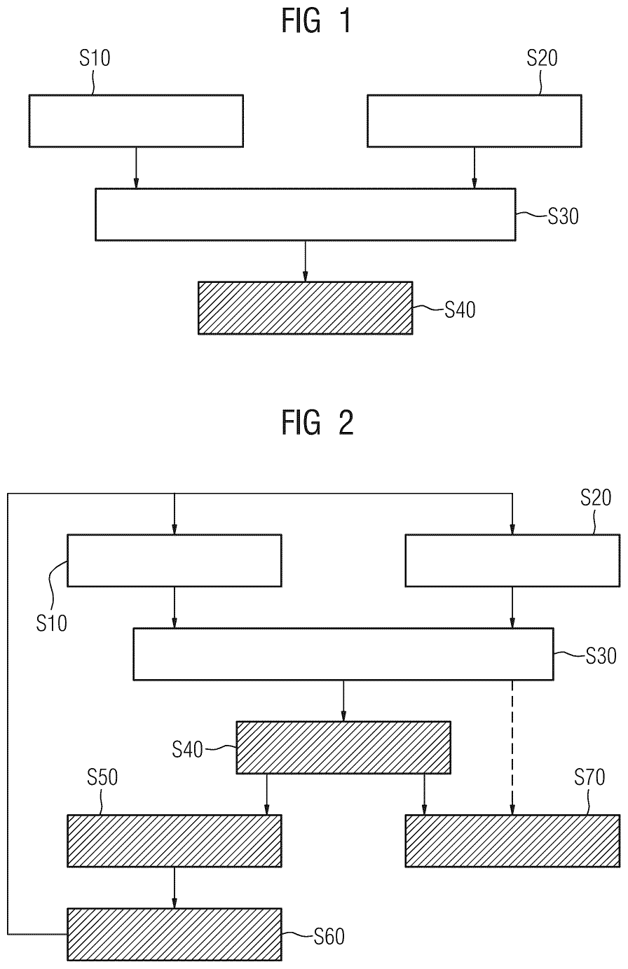

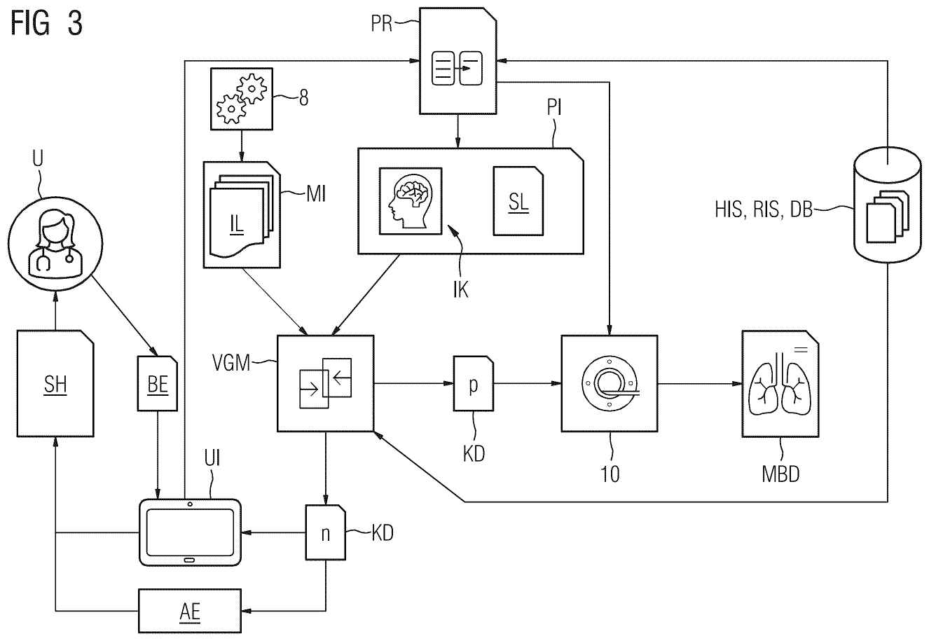

FIG. 1 shows a first exemplary embodiment of a method for ascertaining consistency information KD for a local coil 9 of a magnetic resonance apparatus 10. FIG. 1 here shows the main method steps of the method in a form of embodiment with an optional method step S40 of providing the consistency information, while FIG. 2 additionally illustrates a number of optimal method steps and/or sub steps S40, S50, S60, S70 of the method in one form of embodiment. The methods, or the method steps from FIGS. 1 and 2 are moreover illustrated by the data flow diagram of FIG. 3.

In a method step of an acquisition S10 of measurement information MI of the local coil 9 an actual position IL of the local coil 9 is acquired and provided. The measurement information MI comprises more detailed position information of the local coil, in particular a three-dimensional space description (in x, y and z) of a measurement point in particular starting from a reference point. Preferably the measurement information MI can be represented and provided via a coordinate system. The measurement information MI can in particular be provided to a comparison module VGM. The comparison module VGM can in particular be a processing unit of the magnetic resonance apparatus 10 and/or local coil 9 and/or be comprised by this. The acquisition of the measurement information MI is preferably undertaken with a (Hall) sensor 8 of the local coil 9.

In a method step S20 of ascertaining positioning information PI a required position IL of the local coil 9 is ascertained and provided. The positioning information PI comprises predetermined position information, in particular a required position SL of the local coil 9, in particular with the aid of information IK about a region of the body of the patient. The required position SL of the local coil 9 can, similarly to the actual position IL of the local coil 9, comprise a three-dimensional spatial description (in x, y and z) of a point, in particular starting from a reference point. Preferably the positioning information PI can be shown and provided via a coordinate system. The positioning information PI can in particular be provided to a comparison module VGM.

The ascertaining S20 of positioning information PI can comprise a number of sub steps. The sub steps are not shown in FIGS. 1 and 2, but can be carried out as method steps comprised by S20 one after another and/or in parallel. A sub step can comprise a determination S21 of the required position SL of the local coil 9 with the aid of the predetermined region of the body of the patient. A further sub step can comprise an ascertaining S22 of a patient registration PR. Through the ascertaining S22 of the patient registration PR parameters for an imaging examination via the magnetic resonance apparatus 10 can be determined, set and/or defined. With the aid of the patient registration PR in its turn there can be the ascertaining of the positioning information PI and/or a determination of the required position SL of the local coil 9. The patient registration PR can preferably be determined, derived and ascertained from a hospital information system and/or radiology information system HIS, RIS, DB.

In a method step of ascertaining S30 the consistency information KD with the aid of the positioning information PI and the measurement information MI, a comparison and/or reconciliation preferably occurs of the actual measurement data, the actual position IL with the required position SL of the local coil 9. Here for example, evaluation functions and/or similarity functions can be applied in order to analyze and to quantify the deviations and matches between the data. The ascertaining S30 of the consistency information KD is undertaken in particular by a comparison module VGM comprised by a processing unit. The comparison module VGM can, as a result of the comparison of the measurement information MI with the positioning information PI, output positive p (for example 1) consistency information KD or negative n (for example 0) consistency information KD. The consistency information KD can, as an alternative and/or in addition however, also have a (discrete) value.

The ascertaining S30 of the consistency information KD can in particular comprise a determination of a measure of similarity with a similarity function. The similarity function compares the positioning information PI and the measurement information MI. In this case for example coordinate points of the positioning information PI and the measurement information MI can be compared. In particular the distances of the coordinate points of the positioning information PI and of the measurement information MI can be determined. The measure of similarity depends in this case for example on the sum of the quadratic distances. In particular the measure of similarity depends on the negative sum of the quadratic distances. As an alternative, the measure of similarity can be proportional to the inverse value of the sum of the quadratic distances. In this case it can apply that the smaller the sum of quadratic distances is, the larger is the measure of similarity. In other words the measure of similarity describes to what extent the positioning information PI matches the measurement information MI. As an alternative or in addition the measure of similarity can depend on a cross correlation and/or a normalized cross correlation and/or a covariance and/or a correlation coefficient between the positioning information PI and the measurement information MI.

In an optimal method step of a provision S40 of the consistency information KD the consistency information KD determined is provided. This consistency information KD can be used to ensure that the local coil 9 is correctly positioned and an ascertaining S70 of medical image data of a predetermined region of the body via the local coil and magnetic resonance apparatus takes place. Deviations or anomalies between the positioning information PI and measurement information MI can be recognized by the provision S40 of the consistency information KD to an operator U of the magnetic resonance apparatus 10. The provision S40 of the consistency information KD can for example comprise an output S50 of the consistency information KD via a user interface UI and/or an output unit AE. The consistency information KD can likewise be provided for example to a control unit and/or processing unit of the magnetic resonance apparatus 10 for control of an imaging examination.

In an optimal method step of an output S50 of the consistency information KD, the consistency information KD determined can in particular be output to an operator U. The provision S40 of the consistency information KD can comprise the output of the consistency information KD. The output of the consistency information KD can in particular be carried out in the form of a visual, haptic and/or acoustic notification signal SH. In particular the output of the consistency information KD can be undertaken via a user interface UI and/or output unit AE, in particular to the magnetic resonance apparatus. For example, for negative n consistency information KD the output unit AE of the local coil 9 can send out a notification signal SH to a user U in the form of a colored signal.

The optional step S60 provides for a Human-Machine interaction via a user input. The operator can enter a user input BE via the user interface UI. The user input BE can be processed by the user interface UI or there can be an adaptation and/or modification of the patent registration PR via the user interface UI through the user input BE.

For example the acquisition of the user input can comprise a number of sub steps. In particular a number of different reaction options can be provided, which can be displayed for example in a user screen of the user interface UI. A user input BE can be received by the user interface UI, which is directed to selecting one or more of the reaction options provided and/or to discarding others. The selected reaction options can then be the basis of the modification of the patient registration PR and/or adaptation of the positioning of the local coil.

In accordance with a few implementations the user inputs BE can further be reported back to the analysis function in order to improve this—such as by further training of said function.

The optional step S60 is thus in other words directed to an inclusion of a user input BE. The user input BE can alternatively be entered by a user U in a front-end-processing facility and/or a user screen and be received in a back-end processing facility. The user input BE is preferably an input of the operator U, who makes this entry within the framework of an analysis of the consistency information KD.

The acquisition (and/or ascertaining) of the medical image data S70 essentially comprises an execution of a medical imaging examination via the magnetic resonance apparatus. The acquisition of the medical image data S70 can also comprise subordinate steps, which are necessary for preparation of a medical imaging examination via the magnetic resonance apparatus. During the medical imaging examination medical image data of the part of the body of the patient is recorded according to an imaging protocol.

The method steps of acquisition S10 of measurement information MI and of ascertaining positioning information PI can also be carried out at the same time or in reverse order. The method steps S10, S20, S30 (and S40) can moreover be carried out multiple times and/or iteratively. The method steps S10, S20, S30 (and S40) can moreover in particular be carried out before, but also after and during one (or more) imaging examinations via the magnetic resonance apparatus 10.

FIG. 3 illustrates the data flow of a method for ascertaining consistency information KD for a local coil 9 of a magnetic resonance apparatus 10 in accordance with a form of embodiment.

Measurement information MI of a local coil 9 can be ascertained via a sensor 8. The measurement information MI comprises an actual position IL of the local coil 9 in a magnetic resonance apparatus 10. Position information PI can be determined from a patient registration PR. The position information PI comprises an item of information IK about a region of the body of a patient (to be examined). The position information PI comprises a required position SL of the local coil 9 in the magnetic resonance apparatus 10. The patient registration PR is connected to a database DB. The patient registration PR can interrogate all information from the database DB and/or from a user interface UI needed for carrying out a magnetic resonance examination via the magnetic resonance apparatus 10. The patient registration PR can comprise the initial information for the setting the parameters of the magnetic resonance apparatus 10.

The position information PI and measurement information MI can be received by a comparison module VGM. The comparison module VGM ascertains consistency information KD via a comparison of the position information PI and measurement information MI. The consistency information KD can for example comprise a classification into positive p or negative n. For consistency information KD determined as negative the consistency information KD can be transferred by the comparison module VGM to a user interface UI and/or to an output unit AE. The user interface UI can be comprised for example by the magnetic resonance apparatus 10. The output unit AE can be comprised for example by the local coil 9. The comparison module VGM can moreover be connected to the database DB. For the comparison of the position information PI and measurement information MI the comparison module VGM can interrogate information from the database DB. For example the comparison module VGM can compare the information of the patient registration PR, in particular the positioning information PI, with the information from the database DB.

Via the user interface UI and/or output unit AE a notification signal SH can be output to an operator U of the magnetic resonance apparatus 10. Moreover the entry of a user input BE can be made possible for the operator U of the magnetic resonance apparatus 10 via the user interface UI. The user input BE can be acquired and processed by the user interface UI. Depending on the user input BE, the user interface UI can transfer a signal to the patient registration PR. For example the information IK held in the patient registration PR about the part of the body to be examined and/or the required position SL of the local coil can be adapted by the signal of the user interface UI. In other words the patient registration PR can be adapted by the user interface UI via a user input BE.

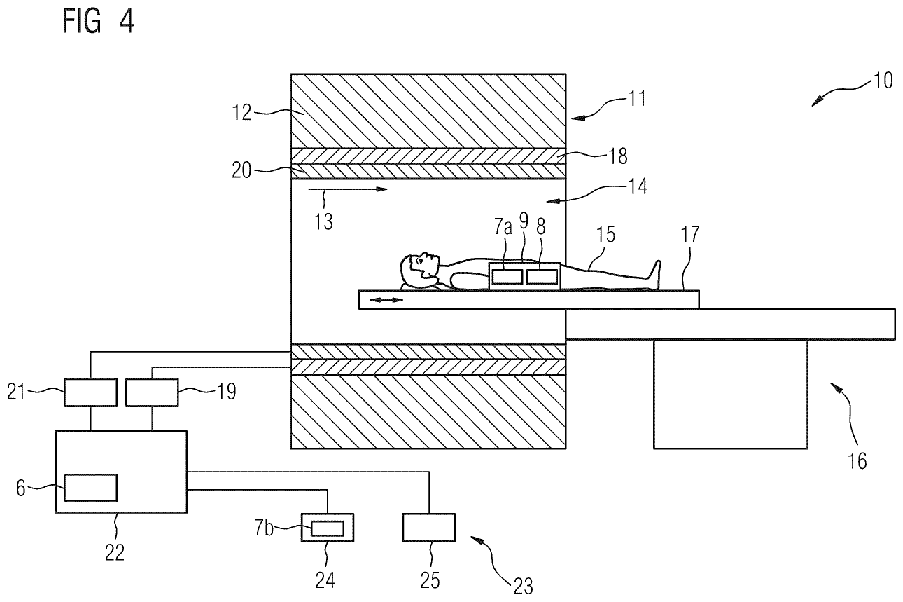

Shown schematically in FIG. 4 is a magnetic resonance apparatus 10. The magnetic resonance apparatus 10 comprises a magnet unit 11, which has a main magnet 12 for generation of a strong and in particular temporally constant main magnetic field 13. Moreover, the magnetic resonance apparatus 10 has a magnet opening 14 for receiving a patient 15, i.e. the magnet opening 14 can be seen as a patient receiving area. The magnet opening 14 is embodied in the form of a cylinder with a central axis z in the z direction and is enclosed in a circumferential direction by the magnet unit 11. The magnet opening 14 is delimited by an inner wall W of the magnet unit 11. The patient 15 can be pushed via a patient support apparatus 16 of the magnetic resonance apparatus 10 into the magnet opening 14. For this, the patient support apparatus 16 has a patient table 17 embodied movably within the magnet opening 14.

The magnet unit 11 furthermore has a gradient coil unit 18 with at least one gradient coil for generation of magnetic field gradients, which are used for spatial encoding during imaging. The gradient coil unit 18 is controlled via a gradient control unit 19 of the magnetic resonance apparatus 10. The magnet unit 11 furthermore comprises a radio frequency antenna unit 20 with at least one transmit antenna, which in the present exemplary embodiment is embodied as a whole body coil permanently integrated into the magnetic resonance apparatus 10. The radio frequency antenna unit 20 is controlled by a radio frequency antenna control unit 21 of the magnetic resonance apparatus 10 and irradiates radio frequency magnetic resonance sequences, in particular RF transmit pulses into a patient receiving area, which is essentially formed by the area of the magnet opening 14 of the magnetic resonance apparatus 10. There is an excitation of atomic nuclei by the main magnetic field 13 generated by the main magnet 12, in that nuclear spins are diverted from their rest position. Through relaxation of the excited atomic nuclei magnetic resonance signals are created. The radio frequency antenna unit 20 can basically be embodied to receive the magnetic resonance signals.

In addition to the radio frequency antenna unit 20 and/or comprised by said unit, the magnetic resonance apparatus can comprise a local coil 9. The local coil 9 can be embodied in this case similarly to the radio frequency antenna unit 20. The local coil 9 can however, by contrast with the radio frequency antenna unit 20, be positioned directly on the patient 15 around a part of the body of the patient 15. The magnet unit 11 can thus furthermore comprise at least one local coil embodied with a receive antenna, which in the present exemplary embodiment is positioned in the magnet opening 14 of the magnetic resonance apparatus 10 on an arm of the patient 15, in particular on the elbow. The local coil 9 can in particular receive radio frequency magnetic resonance sequences, in particular RF transmit pulses, from a patient receiving area. The patient receiving area is essentially formed by the area of the magnet opening 14 of the magnetic resonance apparatus 10. Basically the local coil 9 can be embodied for receiving the magnetic resonance signals or the relaxation of the excited atomic nuclei of the region of the body of the patient 15.

For control of the main magnet 12, the gradient control unit 19 and for control of the radio frequency antenna control unit 21 the magnetic resonance apparatus 10 has a system control unit 22. The system control unit 22 centrally controls the magnetic resonance apparatus 10, such as for example the execution of a predetermined imaging gradient echo sequence. Moreover the system control unit 22 comprises an evaluation unit not shown in any greater detail for evaluation of the magnetic resonance signals that are acquired during the magnetic resonance examination. Furthermore, the magnetic resonance apparatus 10 comprises a user interface 23, which is connected to the system control unit 22. Control information such as for example imaging parameters, as well as reconstructed magnetic resonance images, can be displayed on a display unit 24, for example on at least one monitor of the user interface 23, for a medical operator. The user interface 23 furthermore has an input unit 25, via which information and/or parameters can be entered by the medical operators during a measurement process. The user interface 23, in particular the display unit 24, can moreover comprise an output unit 7b (corresponding to UI from FIG. 3). The output unit 7b can be embodied for output of a visual, haptic and/or acoustic notification signal.

The magnetic resonance apparatus 10, in particular the system control unit 22, can moreover comprise a comparison module 6 (corresponding to VGM from FIG. 3), as is shown schematically in FIG. 3. The comparison module 6 serves to ascertain the consistency information KD. The consistency information is designed to ensure that a different part of the body of the patient 15 than that intended is accidentally recorded with the magnetic resonance apparatus 10. The comparison module 6 can be connected to a sensor 8 of the local coil. The comparison module 6 can be embodied to determine the position of the local coil 9 in the magnet opening 14 with the aid of position information ascertained by the sensor 8.

The comparison module 6 receives the measurement information of the local coil and can determine the positioning information. The comparison module 6 can correspond to a test unit that is connected to the system control unit 22 and the user interface 22. The comparison module 6 can however be completely integrated into the system control unit 22. The comparison module 6 can be embodied to check whether the consistency information adheres to or falls below a limit value. When the limit value is not reached and/or is exceeded, the comparison module 6 can send a warning message to the user interface 23 in order to inform the medical operator.

The magnetic resonance apparatus 10 (not shown) can moreover have a sensor unit, which can be arranged on and/or in the inner wall. The sensor unit can in particular detect measurement information of the local coil, in particular an actual position, and send it to the comparison module 6. The sensor unit is embodied to determine the position of the local coil 9 within the patient receiving area 14 and to send position information to the comparison module 6.

The local coil 9 can also comprise an output unit 7a (corresponding to AE from FIG. 3). In particular the output unit 7a can be embodied for output of a visual, haptic and/or acoustic notification signal. For example the local coil 9 can comprise an LED strip embodied for output of the consistency information, which in particular can output visual notification signals to an operator depending on the consistency information determined.

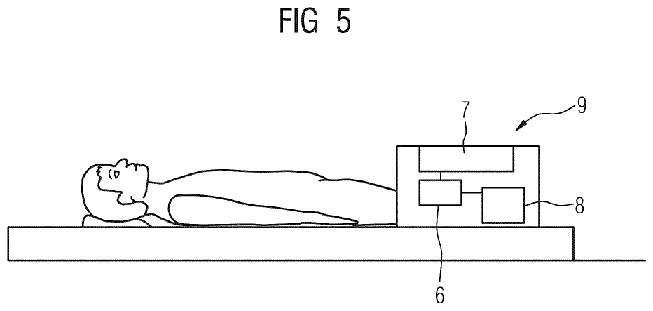

Shown schematically in FIG. 5 is a local coil 9 in one form of embodiment. The local coil 9 shown comprises a sensor 8, a comparison module 6 (corresponding to VGM from FIG. 3) and an output unit 7 (corresponding to AE from FIG. 3). The sensor 8 is embodied for acquiring measurement information of the local coil 9. In particular the sensor 8 of the local coil 9 can determine a relative position (depending on a reference point) of the local coil 9 within a magnetic resonance apparatus. In particular the sensor 8 of the local coil 9 can detect the actual position of the local coil 9, the actual position. The output unit 9 is embodied to output a notification signal to an operator of the magnetic resonance apparatus. The output unit 9 can be embodied for output of a visual, haptic and/or acoustic notification signal. For example the output unit 9 can comprise a sound generation unit for output of an acoustic notification signal. The output unit 9 can for example also comprise a vibration unit for output of a haptic notification signal. The comparison module 6 can be embodied as a processing unit. The comparison module 6 of the local coil 9 can be connected to the sensor 8 and/or the output unit 7 for an exchange of data. For example the comparison module 6 can receive measurement information of the sensor 8 and/or send an output signal for output of a notification signal to the output unit 7. The comparison module 6 can be connected to a control unit of a magnetic resonance apparatus for exchange of data. In particular the comparison module 6 can receive position information, in particular a required position of the local coil 9 from the control unit of a magnetic resonance apparatus. The comparison module 6 of the local coil 9 can in particular be embodied for a comparison of the measurement information with the positioning information and/or for ascertaining consistency information. The comparison module 6 can in particular be embodied for carrying out a method as depicted in FIGS. 1 and 2.

In conclusion it is pointed out once again that the method described in detail above, as well as the magnetic resonance apparatus shown, merely involve exemplary embodiments, which can be modified by the person skilled in the art in a wide variety of ways without departing from the area of the invention. Furthermore, the use of the indefinite article “a” or “an” does not exclude the features concerned also being able to present multiple times. Likewise, the term “unit” does not exclude the components involved consisting of a number of interacting subcomponents, which may possibly can also be spatially distributed. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for ascertaining consistency information for a local coil of a magnetic resonance apparatus, wherein the magnetic resonance apparatus is configured to ascertain medical image data of a predetermined region of a body of a patient through an imaging examination via the local coil, the method comprising:
acquiring measurement information of the local coil, the measurement information including an actual position of the local coil;
ascertaining positioning information, the positioning information including a required position of the local coil; and
ascertaining the consistency information by comparing the measurement information with the positioning information.

2. The method of claim 1, wherein the ascertaining the positioning information comprises determining the required position of the local coil using the predetermined region of the body of the patient.

3. The method of claim 1, further comprising:
ascertaining of medical image data of the predetermined region of the body of the patient by an imaging examination, the imaging examination being based on the consistency information.

4. The method of claim 1, further comprising:
ascertaining a patient registration, the patient registration including parameters for an imaging examination, wherein the ascertaining the positioning information comprises determining the required position of the local coil using the patient registration.

5. The method of claim 4, wherein the ascertaining the patient registration uses information from at least one of a hospital or radiology information system.

6. The method of claim 1, further comprising:
providing the consistency information to an operator of the magnetic resonance apparatus.

7. The method of claim 6, wherein the providing the consistency information comprises outputting at least one of a visual, a haptic or an acoustic notification signal to the operator of the magnetic resonance apparatus.

8. The method of claim 1, wherein the acquiring the measurement information of the local coil is performed by a Hall sensor.

9. A magnetic resonance apparatus configured to ascertain medical image data of a predetermined region of the body of a patient via a local coil, wherein the magnetic resonance apparatus is configured to perform the method of claim 1.

10. A local coil configured to ascertain medical image data of a predetermined region of the body of a patient via a magnetic resonance apparatus, wherein the local coil is configured to perform the method of claim 1.

11. The method of claim 2, further comprising:
ascertaining of medical image data of the predetermined region of the body of the patient by an imaging examination, the imaging examination being based on the consistency information.

12. The method of claim 2, further comprising:
ascertaining a patient registration, the patient registration including parameters for an imaging examination, wherein the ascertaining the positioning information comprises determining the required position of the local coil using the patient registration.

13. The method of claim 2, further comprising:
providing the consistency information to an operator of the magnetic resonance apparatus.

14. The method of claim 2, wherein the acquiring the measurement information of the local coil is performed by a Hall sensor.
